(19) United States
(12) Patent Application Publication
(10) Pub. No.: US 2004/0106165 A1
(43) Pub. Date: Jun. 3, 2004

(54) Screening method for detecting amidase and nitrile hydratase activity and use thereof

(76) Inventors: Christoph Reisinger, Graz (AT); Anton Glieder, Gleisdorf (AT); Helmut Schwab, Graz (AT)

Correspondence Address: WENDEROTH, LIND & PONACK, L.L.P., 2033 K STREET N. W., SUITE 800, WASHINGTON, DC, 20006-1021, US

(21) Appl. No.: 10/619,000

(22) Filed: Jul. 15, 2003

(30) Foreign Application Priority Data
Jul. 15, 2002 (AT) A 1056/2002

(51) Int. Cl. C12Q 1/34; C12Q 1/02

(52) U.S. Cl. 435/18; 435/29

(57) ABSTRACT

Screening method for detecting amidase or nitrohydratase activity, which comprises

a) preparing a replica of amidases- or nitrile hydratase-expressing cell colonies by means of a support, followed by,

b₁) in the case of colonies having nitrile hydratase genes to be assayed, firstly incubating the colonies adhering to the support with a substrate solution composed of a nitrile of the formula (I) R—CN and of a buffer, and subsequently

b₂) carrying out an incubation with a buffered hydroxylammonium salt solution, or,

c) in the case of cell colonies having amidase genes to be assayed, incubating the colonies adhering to said support with a substrate solution composed of an amide of the formula (II) R—CONH₂, a hydroxylammonium salt and a buffer, and then,

d) following b₂) or c), staining the active colonies by transferring the support to an iron (III) salt solution, and

e) where appropriate, isolating the active colonies, and use thereof.

The invention relates to a novel screening method useful for detecting amidase and nitrile hydratase activity at the level of bacterial colonies and to the use of the method for cloning an amidase or a nitrile hydratase gene.

Amidases are especially important for the hydrolysis of sensitive amides for which the drastic conditions of chemical hydrolysis are not applicable. They are also found to be used as enzymes following nitrile hydratases in the hydrolysis of nitriles to carboxylic acids.

While several amidases preferring a wide variety of substrates have already been characterized, there is always a search for enzymes having superior properties. It is state of the art to clone genes coding for amidases and to obtain amidase by expressing these genes in appropriate host cells. The publications of the prior art, for example J. Bacteriol. 1991, 173 (21): 6694-704; J. Bacteriol. 1990, 172 (12): 6764-73; Biochim Biophys Acta. 1991, 16; 1088 (2): 225-33 etc., which relate to cloning of amidases, have chosen the classical path to isolating the gene. This path comprises partial sequencing of the peptide, construction of degenerated oligos, amplification of a probe by means of PCR, and colony hybridization of a gene bank of the organism. Consequently, there is an interest in improving the properties of amidases by protein engineering methods, an important approach thereof being the strategy of “directed evolution”. The usefulness of “directed evolution” and of other mutagenesis strategies is described, for example, in Curr Opin Chem Biol., 1999, 3 (1): 54-9 and Curr Opin Biotechnol. 2001, 12 (6): 545-51.

“Directed evolution” programs for amidases, however, require the use of complicated and cost-intensive analytical methods such as photometry, HPLC, etc., as described, for example, in Enzyme and Microbial Technology 2000, 26: 152-158, since detection of carboxylic acids and ammonium against a biological background is not a simple task. Although it is possible to screen ammonium from amide hydrolysis as sole nitrogen source in the medium, only concentrations are obtainable, since ammonium diffuses quickly and, for example, E. coli has very effective ammonium uptake mechanisms.

Like with amidases, the classical method (protein purification, sequencing, construction of degenerated primers, amplification of a probe by means of PCR and colony hybridization of a gene bank or direct DNA screening) must be referred to as state of the art for cloning nitrile hydratases, which is described, for example, in Biosci. Biotechnol. Biochem., 1993 August, 57:1323-8; Biochim. Biophys. Acta, 1991 December, 1129:23-33 and J. Bacteriol., 1991 Apr, 173:2465-72 etc.

Accordingly, it was the object of the present invention to find a method which enables amidases or nitrile hydratases to be cloned without the aid of sequence information and amidase or nitrile hydratase activity in bacteria or cell colonies to be determined with little time and cost and which furthermore allows rapid comparison of amidase and nitrile hydratase activity of large numbers of colonies of organisms for selection of improved clones and mutants.

Unexpectedly, this object was achieved by a 4- or 5-stage screening method based on calorimetric indication of amidase activity.

Accordingly, the present invention relates to a screening method for detecting amidase or nitrile hydratase activity, which comprises

a) preparing on a suitable support a replica of cell colonies which express amidases or nitrile hydratases and which have grown on a suitable, solidified medium, followed by,

b₁) in the case of colonies having nitrile hydratase genes to be assayed, firstly incubating the cells adhering to the support with a substrate solution composed of a nitrile of the formula (I) R—CN in which R is a C₁-C₂₀-alkyl, C₆-C₂₀-aryl or C₅-C₂₀-heteroaryl radical which is unsubstituted or mono- or poly-substituted with substituents inert under the reaction conditions and of a buffer, and subsequently

b₂) carrying out an incubation with a buffered hydroxylammonium salt solution, or,

c) in the case of cell colonies having amidase genes to be assayed, incubating the colonies adhering to said support with a substrate solution composed of an amide of the formula (II) R—CONH₂, in which R is as defined above, a hydroxylammonium salt and a buffer, and then,

d) following b₂) or c), staining the active colonies by transferring the support to an iron (III) salt solution, and

e) isolating, where appropriate, cells of said active colonies from said support or from the original areas in which said colonies have been present on the solidified medium.

The present screening method of the invention makes it possible to detect amidase activity and, indirectly, nitrile hydratase activity in a simple and cost-effective manner. The principle of the screening method of the invention and of the screening assay used is based on a “secondary activity” exhibited by some amidases which naturally catalyze conversion of amides to carboxylic acids. Examples of these amidases are those of various Rhodococcus strains such as, for example, Rhodococcus sp. R312, Rhodococcus sp. N774, Rhodococcus rhodochrous J 1, etc. or of various Pseudomonas strains such as, for example, Pseudomonas aeruginosa, Pseudomonas chlororaphis B23, etc., which are described, for example, in Appl. Environ Microbiol, 1998 August 64:8 2844-52. Apart from transferring the acyl radical of an amide to water (=amide hydrolysis), they also catalyze the transfer thereof to hydroxylamine in the presence of hydroxylammonium salts to give hydroxamic acids. Hydroxamic acids, in turn, form deep red to purple complexes with iron (III) ions, thereby indicating colorimetrically the formation of said hydroxamic acid and thus the presence of amidase activity (actually “transacylase activity”), as described, for example, in J Gen Microbiol. 1969, 57(2): 273-85 and Anal. Chem., 1952, 24(5), 898-900.

Nitrile hydratases, in contrast, convert nitrites to amides which, as mentioned above, are substrates for amidases and can therefore be detected according to the screening method of the invention.

Carrying out the screening method of the invention can be divided into 4 or 5 steps. In the case of detecting amidase activity, step a) comprises preparing a replica of cell colonies which express amidase genes and which have grown on an appropriate medium such as, for example, a culture plate or on a solidified medium, for example an agar plate. Said replica is prepared by removing or “casting” cell material from the colonies on the plate by means of a suitable support, for example by means of a membrane or a filter paper. In the process, a relatively large part of the cells present in said colonies are transferred to the membrane or the filter paper, preserving the spatial arrangement of the colonies to one another.

Membranes which are suitable here are those which exhibit adequate dimensional stability and adhesion of cells from the colonies and which have appropriate porosity, such as, for example, polyamide membranes or nitrocellulose membranes.

If high resolution is desired, preference is given to using dimensionally stable nitrocellulose membranes which allow, for example, preparation of precise replicas of up to 5 000 colonies on a standard Agar plate (diameter approximately 9 cm). However, if lower resolutions of about 100 colonies are sufficient, preference is given to using the substantially cheaper filter papers.

When preparing the replicas, good adhesion of the cells to the support material is of essential importance. Different host strains used for the cloning, but also different incubation times and conditions can decisively influence this property.

In the next step, i.e. in step c), the cells adhering to the membrane or to the filter paper are then incubated with the substrate solution. In this case, the substrate solution comprises an amide of the formula (II) R—CONH₂, a hydroxylammonium salt and a buffer.

In the formula (II) R is a C₁-C₂₀-alkyl, C₆-C₂₀-aryl or C₅-C₂₀-heteroaryl radical which is unsubstituted or mono- or poly-substituted with substituents inert under the reaction conditions.

C₁-C₂₀-alkyl here means saturated or mono- or poly-unsaturated, linear, branched or cyclic, bridged, primary, secondary or tertiary hydrocarbon radicals, such as, for example, methyl, ethyl, ethenyl, propyl, i-propyl, i-propenyl, butyl, i-butyl, t-butyl, butenyl, butynyl, pentyl, cyclopentyl, i-pentyl, neopentyl, pentenyl, hexyl, i-hexyl, cyclohexyl, cyclohexylmethyl, 3-methylpentyl, 2,2-dimethylbutyl, 2,3-dimethylbutyl, octyl, cyclooctyl, decyl, cyclodecyl, dodecyl, cyclododecyl etc.

Preference is given to C₁-C₁₂-alkyl radicals and particular preference is given to C₁-C₈-alkyl radicals.

The alkyl group may be unsubstituted or mono- or poly-substituted with groups inert under the reaction conditions. Examples of suitable substituents are unsubstituted or substituted aryl or heteroaryl groups such as phenyl, naphthyl or indolyl groups, halogen, hydroxy, C₁-C₆-alkoxy, aryloxy, preferably C₆-C₂₀-aryloxy, C₁-C₆-alkylthio, cyano, amino, ether, thioether, carboxylic ester, sulfoxide, sulfone, sulfonic acid, sulfonic ester, sulfinic acid, mercaptan, nitro or azido groups.

Preferred substituents are unsubstituted or substituted phenyl or naphthyl, halogen, hydroxy, cyano, amino, C₁-C₆-alkoxy, C₁-C₆-alkylthio or phenoxy.

Aryl means preferably C₆-C₂₀-aryl groups such as, for example, phenyl, biphenyl, naphthyl, indenyl, fluorenyl etc.

The aryl group may be unsubstituted or mono- or poly-substituted. Suitable substituents are again unsubstituted or substituted aryl or heteroaryl groups such as phenyl, naphthyl or indolyl groups, halogen, hydroxy, C₁-C₆-alkyl, C₁-C₆-alkoxy, aryloxy, preferably C₆-C₂₀-aryloxy, C₁-C₆-alkylthio, cyano, amino, ether, thioether, carboxylic ester, sulfoxide, sulfone, sulfonic acid, sulfonic ester, sulfinic acid, mercaptan, nitro or azido groups.

Preferred substituents are unsubstituted or substituted phenyl or naphthyl, halogen, hydroxy, cyano, amino, C₁-C₆-alkoxy, C₁-C₆-alkylthio or phenoxy.

Heteroaryl means cyclic radicals containing at least one S, 0 or N atom in the ring. Examples of these are furyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzoimidazolyl, purinyl, carbazolyl, oxazolyl, thiazolyl, isothiazolyl, 1,2,4-thiadiazolyl, isoxazolyl, pyrrolyl, pyrrolidinyl, quinazolinyl, pyridazinyl, phthalazinyl, morpholinyl, etc. If necessary, functional 0 or N groups can be protected.

The heteroaryl group may be unsubstituted or mono- or poly-substituted with the substituents already listed above.

Examples of suitable substrates are accordingly acetamide, propionamide, butyramide, isobutyramide, valeramide, pivalamide, hexanoamide, acrylamide, methacrylamide, benzamide, nicotinamide, isonicotinamide, lactamide, alaninamide, leucinamide, methioninamide, phenylalaninamide, prolinamide, threoninamide, mandelamide, 4-cyano-3-hydroxybutyramide etc.

Suitable hydroxylammonium salts are chlorides, sulfates, and salts of any other anions which behave neutrally with respect to the reaction. Preference is given to using the commercially common hydroxylammonium chloride.

Examples of suitable buffers are phosphate buffers such as, for example, potassium phosphate or sodium phosphate.

The incubation solution should contain 0.5-2 mol/l, preferably 0.8 to 1.5 mol/l hydroxylammonium salt in from 50 to 200 mM, preferably 80 to 150 mM buffer.

The overall pH of the solution must be adjusted according to the requirements of the specific amidase.

Preference is given to working at a pH in the range from pH 7 to 8.

The amide should be used at concentrations as high as possible, the amount being influenced by the cost factor and solubility. Preferably, concentrations of ≧10 mM should be present, with amides having poor substrate properties being preferably added at substantially higher concentrations of up to 1M.

Where appropriate, it is possible to increase solubility by adding a cosolvent, preferably from the group consisting of DMSO, DMF and C₁-C₄-alcohol, in an amount of up to 20% of the final concentration. Depending on the amidase, this value may even be exceeded.

Depending on the reactivity of the amides and the enzyme activity, the incubation time can range from a few minutes to several hours, shorter incubation times of from 5 minutes up to 30 minutes being preferred.

The incubation temperature is chosen depending on the optimum of the amidase used and is preferably maintained at approx. 20-40° C., usually around 30° C.

The substrate solution is preferably prepared by mixing a buffered hydroxylammonium salt solution with a solution of the desired amide in water or a water-cosolvent mixture.

When using polyamide or nitrocellulose membranes, these may moreover be placed directly on a drop of the substrate solution, with the colonies facing up. Filter papers require large volumes and are soaked with the substrate solution.

During incubation, the corresponding hydroxamic acid is produced locally restricted in spatial proximity to active colonies.

The incubation is followed by staining the active colonies according to step d). For this purpose, the membrane or the filter paper is transferred to a iron (III) salt solution, in a similar manner as described for the substrate solution.

Staining takes place immediately after contacting of the iron (III) salt solution with the hydroxamic acid.

Iron (III) salts which are suitable here are common salts such as, for example, ferric chloride, ferric sulfate, ferric nitrate, etc.

However, a disadvantage of simple iron (III) salts is their sensitivity to hydrolysis at neutral or alkaline pH, which may result in the formation of insoluble and strongly colored hydrate and hydroxide precipitates which may obscure or prevent the color reaction.

In order to prevent this, strongly acidic iron (III) salt solutions, for example ferric chloride in HCl, may be used as color reagent. The disadvantage here is that the bacterial or cell colonies die in the acidic environment, and, as a result, the cells can only be obtained by localization and removal from the original plate.

Therefore, preference is given to using complex iron salts such as, for example, ferric ammonium citrate, ferric ammonium sulfate, etc. in water, which remain still in solution in the neutral pH range.

Staining follows immediately after contact with the staining reagent and usually lasts several minutes (depending on the amount of hydroxamic acid produced).

If membranes are used, these may again be placed directly on a drop of the staining solution, with the colonies facing up.

Filter papers contain a correspondingly larger amount of substrate solution and must therefore be stained with strongly acidified iron (III) salt solutions or the complex iron (III) salt solutions, in order to prevent formation of the hydroxide precipitates. In order to reduce the amount of substrate solution in the filter paper, the latter may be placed on dry filter paper prior to staining. Alternatively, the staining solution may be applied to the relevant areas directly from above.

According to step e), cells of active, now stained colonies may, where appropriate, be isolated from the membrane or the filter paper and used to inoculate suitable nutrient media for further propagation. It is also possible to isolate cells from the original areas on the original plate or the solidified medium on which the colonies have been located.

However, preference is given to isolating the cells of active clones from the transferred and stained colonies on the membranes or filter papers by direct “picking”.

If high colony densities are used for screening, it is advantageous to purify the picked clones via thinning-out smears and renewed screening.

FIG. 1 depicts in diagrammatic form the screening method for detecting amidase activity.

[image]

As described above, nitrile hydratases convert nitriles to amides. Since amides are substrates for amidases, they may be detected using the above-described screening method of the invention. According to the invention, nitrile hydratase activity is determined indirectly with the aid of an amidase via the amide produced.

When detecting nitrile hydratase activity, the variable parameter therefore is no longer the amidase activity but the availability of the substrate, i.e. the amide R—CONH₂, which is obtained by converting a nitrile of the formula (I) R—CN, in which R is as defined above, by means of nitrile hydratases.

In order to determine nitrile hydratase activity via the amide produced, it is possible to add an amidase to the screening solution.

However, a substantially more advantageous method for identifying genes coding for nitrile hydratase is the use of a host strain expressing an appropriate amidase. If DNA coding for nitrile hydratase is introduced into such a host strain in a suitable manner (for example via a plasmid vector), so that expression of the nitrile hydratase gene is possible, nitrile hydratase activity can be recorded directly when using the screening system of the invention. It was therefore also an object of the present invention to find a suitable way of developing a host strain of this kind. The preferred solution found was to use as hosts recombinant E. coli strains which have an amidase gene integrated into the chromosome and which express amidase and produce the enzyme therefrom.

Chromosomal integrations of this kind may be prepared according to different methods, as described, for example, in J. Bacteriol. 1997, 179, (20): 6228-6237 (pKO3), J. Bacteriol. 1989, 171(9): 4617-4622 (pSC101), J. Bacteriol. 2000, 182(8): 2336-2340 (lambda-red) or J. Bacteriol. 1984, 159(2): 783-786 (linear fragments).

Vectors, preferably plasmids, which contain nitrile hydratase genes are introduced (e.g. by transformation) into cells of such recombinant E. coli strains having amidase activity, so that it is possible to detect nitrile hydratase activity, contained in colonies of recombinant clones produced, with the aid of the previously introduced amidase, now “endogenous to bacteria” (as symbolized in FIG. 2).

[image]

Apart from such recombinant E. coli strains, it is also possible to use any other host strains which have strong amidase (or acyl transferase) activity in the absence of nitrile hydratase activity.

In this variant for the screening method of the invention, nitrile hydratases may be detected by cloning DNA from any organism (e.g. after preparation of gene banks) by using a host strain expressing an appropriate amidase. Furthermore, said method of the invention makes it possible to screen for improved nitrile hydratase variants, for example when using strategies of “directed evolution”.

A nitrile hydratase may furthermore be found in those strains (for example naturally isolated strains) which have both nitrile hydratase activity and amidase activity, as can be found, for example, in many bacteria, frequently, for example, in representatives of the genus Rhodococcus.

For this purpose, a replica of cell colonies with nitrile hydratase activity to be assayed, which have grown on a culture plate or a solidified medium, for example an agar plate, is prepared in step a), similarly to the procedure for detecting amidase activity.

Subsequently, the cells adhering to the membrane or the filter paper are incubated with the substrate solution in step b₁). In this case, the substrate solution comprises a nitrile of the formula (I) R—CN, in which R is as defined above, and a buffer. As previously with the amides, the nitrile concentration should be as high as possible, with the exception of those cases in which there is substrate inhibition, high toxicity or an adverse effect of the nitrile on the amidase, with concentrations of 50 to 200 mM being preferred.

A suitable buffer here is any buffer in which nitrile hydratase activity can be expected and which does not irreversibly destroy the amidase present. Preference is given to using phosphate buffers at a concentration of 10-500 mM.

The pH can range from 2 to 9, preference, however, is given to the pH optimum of nitrile hydratase, which is usually close to pH 7.

The reaction time depends on the type of substrate and of nitrile hydratase and ranges from less than 1 min to more than 1 h, and is preferably in the range of 10-20 min. The temperature to be used is the optimum for the enzyme, typically between room temperature and 40° C., frequently approx. 25-30° C.

If the nitrile is poorly soluble, cosolvents, preferably from the group consisting of DMSO, DMF and C₁-C₄-alcohol, may be used, provided that the nitrile hydratase and amidase activities will be preserved.

In this step, nitrile hydratase-active cells produce the corresponding amide of the formula (II) R—CONH₂.

Since amidases usually act slower than nitrile hydratases, the amide is not yet further hydrolyzed completely to the carboxylic acid during this period.

In step b₂), incubation with a buffered hydroxylammonium salt solution is then carried out.

Suitable hydroxylammonium salts are again chlorides, sulfates and any common anions which do not directly interfere with the reaction. Preference is given to the commercially common hydroxylammonium chloride.

Examples of suitable buffers are phosphate buffers such as, for example, potassium phosphate or sodium phosphate, as well as most of the other customary buffers. The incubation solution should contain 0.5 to 2 mol/l, preferably 0.8 to 1.5 mol/l hydroxylammonium salt in 50 to 200 mM, preferably 80 to 150 mM, buffer.

The overall pH of the solution must be adjusted to the requirements of the integrated amidase and is preferably in the range from pH 7 to 8. The incubation time also depends on the substrate/enzyme combinations used. Typically, incubation times of 10-30 min are preferred, in order to prevent diffusion from advancing too far. Cosolvents are usually not required in this screening stage, since the solubility of the amide forming is usually well above that of the nitrile from which it is derived.

The amidase then transfers the acyl radicals from the amide produced to the hydroxylamine and thus generates a hydroxamic acid which is stained in step d) with an iron (III) salt solution, as described above.

Step e) is likewise carried out as described above.

FIG. 3 illustrates the screening method for detecting nitrile hydratase activity:

[image]

The screening method of the invention at the colony level (colony assay) may be used in various ways for detecting amidase activity or nitrile hydratase activity.

Thus, for example, it is possible to use said method for detecting said amidase activity in genomic or cDNA gene banks. Suitable starting organisms are in principle any organisms, for example bacteria of the genus Rhodococcus, such as, for example, Rhodococcus equi or Rhodococcus ruber, the genera Pseudomonas or Acinetobacter, and any other organisms which contain genes of the desired type. It is required for these genes to be expressed in the host strains used.

Furthermore, it is possible to use the screening method of the invention for detecting increased amidase activity and increased nitrile hydratase activity and stability in gene banks containing genes coding for mutants or recombinants of such enzymes (as prepared, for example, during the course of strategies of “directed evolution”). Another possible application is in standard clonings of amidase or nitrile hydratase genes, such as, for example, rapid identification and possible isolation of amidaseor nitrile hydratase-active clones in recloning procedures common in laboratories, for example from a transformation mixture containing a large number of inactive cells, possibly resulting in saving a lot of time and material. It is possible, for example, to use the screening method of the invention for isolating nitrile hydratase genes from gene banks which [lacuna] in recombinant E. coli host strains of the invention by using suitable vectors, for example plasmids.

Further advantages of the screening method of the invention or the “colony assay” are the possibility of high throughputs and the low cost factor, and also the applicability to heterologously expressing clones and also, with described restrictions for nitrile hydratase screening, to wild type organisms.

According to the invention, the screening method of the invention may be used for cloning an amidase gene or a nitrile hydratase gene.

Many of the amidases and nitrile hydratases described in the literature were isolated from bacteria of the genus Rhodococcus, Pseudomonas or Acinetobacter. These frequently exhibit useful properties for biocatalysis so that demand for amidase clones or nitrile hydratase clones continues to increase.

Accordingly, it was another object of the present invention to find a possibility of cloning amidases and nitrile hydratases, which works without the restrictions of the homology methods (PCR, hybridization techniques) or the complicated route of protein purification and protein sequencing.

This object was achieved by the screening method of the invention and by the colony assay of the invention.

Accordingly, the present invention further relates to the use of the above-described screening method for detecting amidase activity in the cloning of an amidase gene or of a nitrile hydratase gene from organisms, preferably from bacteria such as, for example, Rhodococcus, Pseudomonas or Acinetobacter bacteria.

For this purpose, firstly a chromosomal gene bank of the appropriate strain, for example a Rhodococcus strain, is prepared according to the prior art. In this connection, Rhodococcus equi, for example, is grown on a suitable complete medium and (A) the chromosomal DNA is isolated according to a standard method. Suitable methods are any methods which provide clean DNA fractions of high molecular weight, including the commercially available DNA isolation kits.

Subsequently, the chromosomal DNA is (B) partially digested with a restriction enzyme so as to obtain fragments having a suitable size, preferably of about 2-10 kb, in the case of bacteria. These fragments are then, where appropriate after purification, for example via preparative gel electrophoresis or by means of saccharose density gradient centrifugation (C) cloned into appropriate vectors, preferably plasmids, such as, for example, into BamHI-linearized, dephosphorylated pBluescript SK plasmids. A suitable plasmid is in principle any autonomously replicated plasmid construct which permits expression of genes contained therein from promoters belonging to said plasmid or said gene. This is followed by (D) incorporation (for example by transformation) into suitable host cells such as, for example, competent Escherichia coli cells, thereby producing a gene bank with ten thousands of clones which are preserved.

Nitrile hydratases are cloned by carrying out in (D) a transformation into suitable host cells such as, for example, competent E. coli cells, which contain a chromosomal integration of an amidase, for example an amidase of a Rhodococcus species.

In the next step, the gene bank is screened for amidase- or nitrile hydratase-active clones.

For this purpose, preferably dilutions of said gene bank are plated out on suitable solidified media, for example agar plates, and incubated for some time, thereby obtaining plates with colonies, preferably in the range of 1 000-3 000 colonies per plate.

According to the screening system of the invention, a replica of said colonies is prepared in step a), which is incubated according to step b₁) and b₂) (nitrile hydratases) or according to step c) (amidases) with the substrate solution and then stained according to step d). This is followed by isolating according to step e) positive or active clones, for example by removing them using sterile toothpicks.

Due to the high colony density, it is advantageous to culture thinning-out smears and to apply the screening method again, thereby obtaining pure cultures of the positive or active amidase or nitrile hydratase clones.

Finally, the clones can be assayed for the size of their insert by means of restriction analysis and, if required, the DNA section necessary for the enzyme function can be reduced to a minimum size by subcloning, it being possible to use again the screening method of the invention for selecting clones expressing active enzyme.

Thus it is possible to clone a gene coding for an amidase or a nitrile hydratase with the aid of the screening method of the invention and the colony assay of the invention and entirely without the aid of sequence information (for example derived from homologies or peptide sequencing) in a short time. The assay is also very useful for identifying positive clones in the support of simple cloning work.

The gene bank clone as described above may then be sequenced, and the open reading frame can be amplified with suitable primers by means of PCR and cloned via primer-specific cleavage sites into a vector suitable for overexpression, thereby obtaining plasmids which allow strong overexpression of the enzyme in a suitable organism such as, for example, E. coli.

Furthermore, the screening method of the invention allows detection of grades of different activity levels of amidases and nitrile hydratases in cell colonies. In this connection, more intensive staining correlates with a higher activity level. Furthermore, it is possible to adjust the detection windows to the activities given by varying the incubation times during steps b₁ and c, respectively. This procedure allows utilizing the screening method as selection method for, for example, “directed evolution” experiments or other mutagenesis or recombination experiments for obtaining improved variants of amidases and nitrile hydratases by discriminating between more active and less active mutants. The method of the invention may thus be utilized, for example, for isolating more active or more stable clones from gene libraries (e.g. “random mutagenesis” libraries, “gene shuffling” libraries) coding for enzyme variants.

### EXAMPLE 1

Generation of a Chromosomal Gene Bank of Rhodococcus equi IF03730

Rhodococcus equi was grown on Luria broth (LB)-agar medium and chromosomal DNA was obtained according to a standard method, as described in Ausubel et al., Current Protocols, Wiley and Sons, 2002 (Lysozyme/SDS/CTAB disruption, CIP extraction, isopropanol precipitation). The chromosomal DNA was then partially digested with the restriction enzyme Sau3AI so that fragments of 2-10 kb in size were obtained. These fragments were then purified via preparative gel electrophoresis and ligated into BamHI-linearized, dephosphorylated pBluescript SK plasmids. Transformation into electrocompetent Escherichia coli TOP10F′ cells by electroporation resulted in a gene bank with 70 000 clones which were preserved.

Screening of the Gene Bank for Amidase-Active Clones

Gene bank dilutions were plated out on LB-agar plates supplemented with 100 mg/l ampicillin (LB-Amp plates) and incubated overnight, resulting in approx. 2 000 colonies per plate. Cells of these colonies were removed using nitrocellulose membranes (NC-extra 0.2 μm Sartorius) and incubated in the substrate solution (1M hydroxylammonium chloride, 100 mM potassium phosphate, 2.5% acetamide; pH=7.5) at 37° C. for 20 min. The incubated membranes were then stained with a 1M ferric ammonium citrate solution and positive clones were removed from the membrane by means of sterile toothpicks.

The thinning-out smears cultured overnight were again assayed, in order to obtain finally pure cultures of the positive clones. The clones were subsequently assayed for the size of their inserts by means of restriction analysis (cleavage with EcoRI and Xbal). pBS_IFO_amd_{—}3 had a relatively small insert of only 2.5 kb. Plasmid-DNA of this clone was isolated and used for sequencing of said insert.

Amidase Gene Subcloning from pBluescript Vector into Overexpression Vector pMS470Δ8

Sequencing of the gene bank clone finally provided an open reading frame which had homologies to known amidases (Brevibacterium sp. R312). This open reading frame was amplified with primers (rh_equi_lFO_amd3_μl and _r1) by means of PCR (Pwo polymerase=proofreading enzyme) and cloned via the primer-specific cleavage sites (Ndel and Sphl) into the prepared pMS470A8 vector (Balzer, D., G. Ziegelin, W. Pansegrau, V. Kruft, and E. Lanka, 1992. KorB protein of promiscuous plasmid RP4 recognizes inverted sequence repetitions in regions essential for conjugative plasmid transfer. Nucl. Acids Res. 20: 1851-1858), after cleaving with the same enzymes and obtaining the large vector fragment. The plasmid thus obtained (pMS470-48_{—}2_{—}1) was transformed into Escherichia coli BL21 and allowed strong overexpression of the enzyme in E. coil, inducible by IPTG or lactose.

1. A screening method for detecting amidase or nitrile hydratase activity, which comprises
a) preparing on a suitable support a replica of cell colonies which express amidases or nitrile hydratases and which have grown on a suitable, solidified medium, followed by,
b₁) in the case of colonies having nitrile hydratase genes to be assayed, firstly incubating the cells adhering to the support with a substrate solution composed of a nitrile of the formula (I) R—CN in which R is a C₁-C₂₀-alkyl, C₆-C₂₀-aryl or C₅-C₂₀-heteroaryl radical which is unsubstituted or mono- or poly-substituted with substituents inert under the reaction conditions and of a buffer, and subsequently
b₂) carrying out an incubation with a buffered hydroxylammonium salt solution, or,
c) in the case of cell colonies having amidase genes to be assayed, incubating the colonies adhering to said support with a substrate solution composed of an amide of the formula (II) R—CONH₂, in which R is as defined above, a hydroxylammonium salt and a buffer, and then,
d) following b₂) or c), staining the active colonies by transferring the support to an iron (III) salt solution, and
e) isolating, where appropriate, cells of said active colonies from said support or from the original areas in which said colonies have been present on the solidified medium.

2. The screening method as claimed in claim 1, wherein the support used is membranes or filter paper.

3. The screening method as claimed in claim 1, wherein in the substrate solutions in step b₁) nitriles of the formula (I) R—CN and in step c) amides of the formula (II) R—CONH₂ are used, where R is a saturated or mono-unsaturated, linear, branched or cyclic C₁-C₁₂-alkyl radical or a phenyl, biphenyl or naphthyl radical or a pyrrolidinyl radical, which may be unsubstituted or mono- or poly-substituted with substituents from the group consisting of halogen, hydroxy, cyano, amino, unsubstituted or substituted phenyl or naphthyl, C₁-C₆-alkyl, C₁-C₆-alkoxy, C₁-C₆-alkylthio or phenoxy.

4. The screening method as claimed in claim 1, wherein the hydroxylammonium salt used is a chloride or a sulfate.

5. The screening method as claimed in claim 1, wherein the buffer used is phosphate buffer.

6. The screening method as claimed in claim 1, wherein the incubation solutions in step b₂) and c) contain 0.5-2 mol/l hydroxylammonium salt in from 50 to 200 mM buffer.

7. The screening method as claimed in claim 1, wherein, where appropriate, a cosolvent of the group DMSO, DMF or C₁-C₄-alcohol is added to the substrate in step b₁) and in step c).

8. The screening method as claimed in claim 1, wherein the iron (III) salt solutions used in step d) are strongly acidified ferric chloride, ferric sulfate or ferric nitrate solutions or solutions of complex iron (III) salts from the group consisting of ferric ammonium citrate and ferric ammonium sulfate.

9. The screening method as claimed in claim 1, wherein an amidase is added to the screening solution in step b₁) for detecting nitrile hydratase activity.

10. The screening method as claimed in claim 1, wherein an amidase chromosomally integrated into an E. coli strain is used for detecting nitrile hydratase activity expressed from vectors.

11. The use of the screening method as claimed in claim 1 for detecting amidase activity when cloning an amidase gene from appropriate organisms.

12. The use of the screening method as claimed in claim 1 for detecting amidase activity when cloning an amidase gene from bacteria.

13. The use of the screening method as claimed in claim 12, wherein the bacteria used are Rhodococcus, Pseudomonas or Acinetobacter bacteria.

14. The use of the screening method as claimed in claim 1 for detecting nitrile hydratase activity when cloning a nitrile hydratase gene from appropriate organisms.

15. The use of the screening method as claimed in claim 1 for detecting nitrile hydratase activity when cloning a nitrile hydratase gene from bacteria.

16. The use of the screening method as claimed in claim 15, wherein the bacteria used are Rhodococcus, Pseudomonas or Acinetobacter bacteria.

17. The use of the screening method as claimed in claim 1 for detecting increased amidase activity in random mutagenesis libraries.

18. The use of the screening method as claimed in claim 1 for detecting increased nitrile hydratase activity and stability in random mutagenesis libraries.

19. The use of the screening method as claimed in claim 1 for isolating nitrile hydratase genes from gene banks in E. coli plasmids.
